(19) United States
(12) Patent Application Publication
(10) Pub. No.: US 2011/0152741 A1
(43) Pub. Date: Jun. 23, 2011

(54) CANNULA SYSTEM

(76) Inventors: Michael Banchieri, Discovery Bay, CA (US); Tamer Ibrahim, Pleasant Hill, CA (US)

(21) Appl. No.: 12/966,924

(22) Filed: Dec. 13, 2010

Related U.S. Application Data
(60) Provisional application No. 61/288,763, filed on Dec. 21, 2009.
(60) Provisional application No. 61/288,752, filed on Dec. 21, 2009.
(60) Provisional application No. 61/288,614, filed on Dec. 21, 2009.

(51) Int. Cl. A61B 17/34; A61M 1/36

(52) U.S. Cl. 604/6.16; 604/164.13; 604/500

(57) ABSTRACT

A cannula system for removing fluid from a patient's vasculature includes a cannula and an obturator. The cannula includes a proximal end, a distal end, and an elongate flexible tube extending therebetween. The cannula has a central lumen. The obturator is sized for insertion into the cannula's central lumen and is also configured to extend beyond the cannula distal end. A tapered portion at the cannula's distal end provides a smooth transition and an interference fit between the cannula and the obturator. The obturator includes an obturator central lumen and an opening at its distal end that is sized to receive a guidewire.

### CROSS-REFERENCE TO RELATED APPLICATION

This application claims the benefit of U.S. Provisional Application No. 61/288,763, filed Dec. 21, 2009, the entirety of which is hereby incorporated by reference. This application is also related to U.S. Provisional Application Nos. 61/288,752 and 61/288,614, which are incorporated by reference in their entireties, herein.

### SUMMARY

A cannula system for use in minimally invasive cardiac surgery includes a cannula and an obturator. The cannula system can also include an obturator, guidewire, stylet, and/or dilator system. The dilator system includes dilators of increasing diameter. Each dilator is configured to dilate an access vein or other portion of a patient's vasculature. For example, each dilator is configured to dilate the femoral vein at the location of the groin through a percutaneous approach via fascial tissue.

The dilator is selected to have a predetermined stiffness. Dilator stiffness may be controlled by varying its wall thickness, or by using stiffer materials (e.g., plastics, polymers, reinforced plastics, etc.). The dilator's stiffness provides column strength useful to advance the cannula through the patient's fascial tissue in a percutaneous approach, into and through the vasculature. In another embodiment, a more compliant, flexible dilator may be used with an accessory stylet, where the stylet provides the desired stiffness and column strength.

The obturator is insertable into the cannula, and when fully inserted, extends beyond the cannula's distal end. The cannula is designed to be conforming, and to have a slight interference fit with a portion of the obturator. The cannula does not have an interference fit with the obturator along the entire cannula length, but merely along just a portion of the cannula's elongate body. For example, the cannula can have an interference fit with the obturator only at the distal portion of the cannula's elongate body. Such a configuration allows the obturator to be easily inserted and removed from the cannula's central, internal lumen.

In addition, a taper at the cannula's distal end provides a smooth transition from the cannula to the obturator, and avoids creating a ledge or other surface that could become snagged during insertion of the cannula system in the patient's vasculature. The cannula's distal end, or tip, is tapered to a thin wall thickness and softened material such that when the cannula system is bent (for example, during advancement through a patient's tortuous vasculature) the cannula and obturator maintain a conforming geometry, and a fish-mouthing phenomenon is avoided. For example, the distal end of the cannula remains tightly in contact with the outside surface of the obturator as the cannula system is bent and advanced through the patient's vasculature.

The cannula body can include an axial taper to minimize the cannula's insertion profile at its distal end. The cannula body can also include larger inside diameter at the cannula's proximal end, which can enhance the blood flow characteristics therethrough. The cannula body can have a thick- or thin-wall configuration. In a thin-wall configuration (which may provide desirable flexibility and maneuverability) the cannula body can include various reinforcing elements extending longitudinally therethrough. For example, the cannula's wall can include a wound wire, spring, or be made of discrete sections of material having a greater stiffness than the overlying canula material.

The obturator has a tapered distal end to facilitate insertion and advancement through the patient's vasculature. The distal tip of the obturator can include a small hole configured to receive and to be relatively conforming to a guidewire. The obturator is configured to be advanced over a guidewire. In one embodiment, the obturator is colored blue to designate venous return.

### BRIEF DESCRIPTION OF THE DRAWINGS

FIG. 1 illustrates a cannula system in accordance with one embodiment;

FIG. 2 illustrates a cross-sectional side view of the cannula system of FIG. 1; and

FIG. 3 is a flow chart illustrating one method of venous cannula insertion into the femoral vein of a medical patient, suitable for use with a cannula system, including the embodiments of FIGS. 1 and 2.

### DETAILED DESCRIPTION

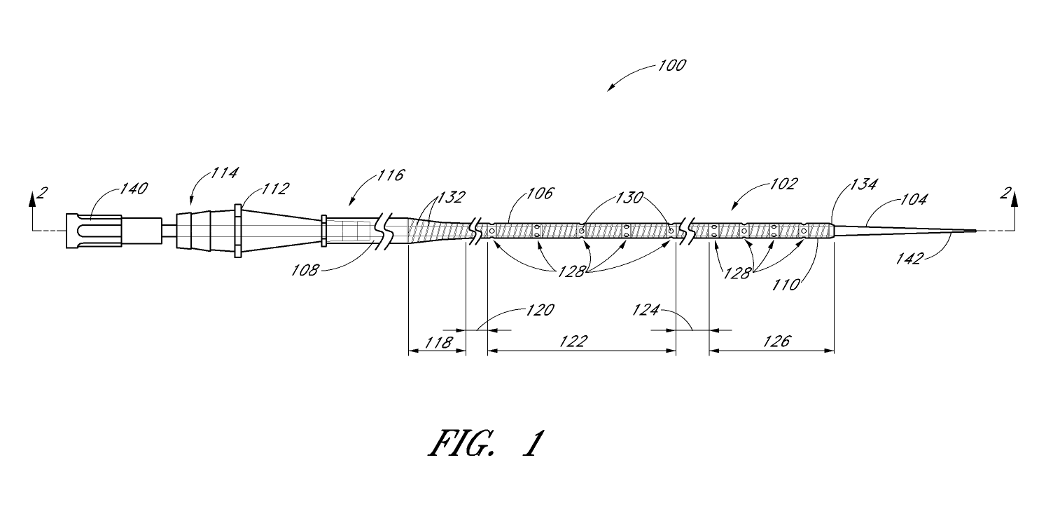

FIG. 1 illustrates one embodiment of a cannula system 100 configured for delivery into the vasculature of a medical patient. The cannula system 100 may be used to remove fluids (e.g., blood, deoxygenated blood, etc.) from or to deliver fluids (e.g., blood, oxygenated blood, drugs and/or medications, etc.) to the medical patient's body. In one embodiment, the cannula system 100 is used as a femoral venous cannula, and is configured as a conduit to deliver deoxygenated blood from a patient's vasculature to an external cardiopulmonary bypass device.

The cannula assembly 100 includes a cannula 102 and an obturator 104 specifically designed to mate with the cannula 102, as discussed below. The cannula 102 includes an elongate flexible tube 106, which is often constructed from a polymer or other flexible, biocompatible material. The elongate tube 106 has sufficient flexibility for percutaneous delivery into a patient's vasculature, while maintaining sufficient wall strength to resist compression and kinking.

The elongate tube 106 extends from its proximal end 108 to its distal end 110. The proximal end 108 has an enlarged diameter compared to the remaining portion of the elongate tube 106, and is sized to attach to the distal end of a connector 112. The connector's proximal end 114 is barbed, and sized to removably attach to the tubing of a cardiopulmonary bypass machine. In one embodiment, the barbed proximal end 114 of the connector 112 is sized to attach to tubing having an inside diameter of 0.5″ (1.3 cm).

The proximal end 108 of the cannula's elongate tube 106 includes an unreinforced region 116. The unreinforced region 116 is able to be squeezed or clamped closed, for example by using a surgical clamp. Clamping the unreinforced region 116 is useful for temporarily preventing fluid flow through the cannula 102. For example, during insertion or removal of the cannula assembly into a patient's vasculature, it is often advantageous to temporarily prevent fluid flow through the cannula 102, as discussed in further detail below.

The unreinforced region 116 transitions in diameter over a tapered segment 118 to the smaller outside diameter of the remaining portion of the cannula's flexible elongate tube 106. At least one substantially cylindrical, tubular segment extends from the tapered segment 118 to the cannula's distal end 110. In one embodiment, the cannula's flexible elongate tube 106 includes four segments, i.e., first, second, third and fourth segments 120, 122, 124, 126, as illustrated in FIG. 1. In other embodiments, the canula's flexible elongate tube 106 includes only three, two, or just one segment between the tapered segment 116 and the distal end 110.

Each segment 120, 122, 124, 126 can have a substantially cylindrical shape, and a different inside diameter, outside diameter, and/or wall thickness, than each other segment 120, 122, 124, 126. For example, in one embodiment, the first segment's 120 inside diameter is slightly larger than the second segment's 122 inside diameter, which is slightly larger than the third segment's 124 inside diameter, which is slightly larger than the fourth segment's 126 inside diameter. The segments' 120, 122, 124, 126 outside diameters can decrease in a similar manner, as well.

In one embodiment, the inside diameters of the first, second and third segments 120, 122, 124 can be sized slightly larger than the outside diameter of the obturator 104, while the inside diameter of the fourth segment 126 can be sized slightly smaller than the outside diameter of the obturator 104. Sizing only the fourth segment 126 (or the distal-most segment in embodiments having more or fewer than four segments) advantageously allows the distal end 110 of the cannula 102 to securely mate with the obturator 104, while providing reduced friction to allow easier insertion and removal of the obturator from the cannula 102.

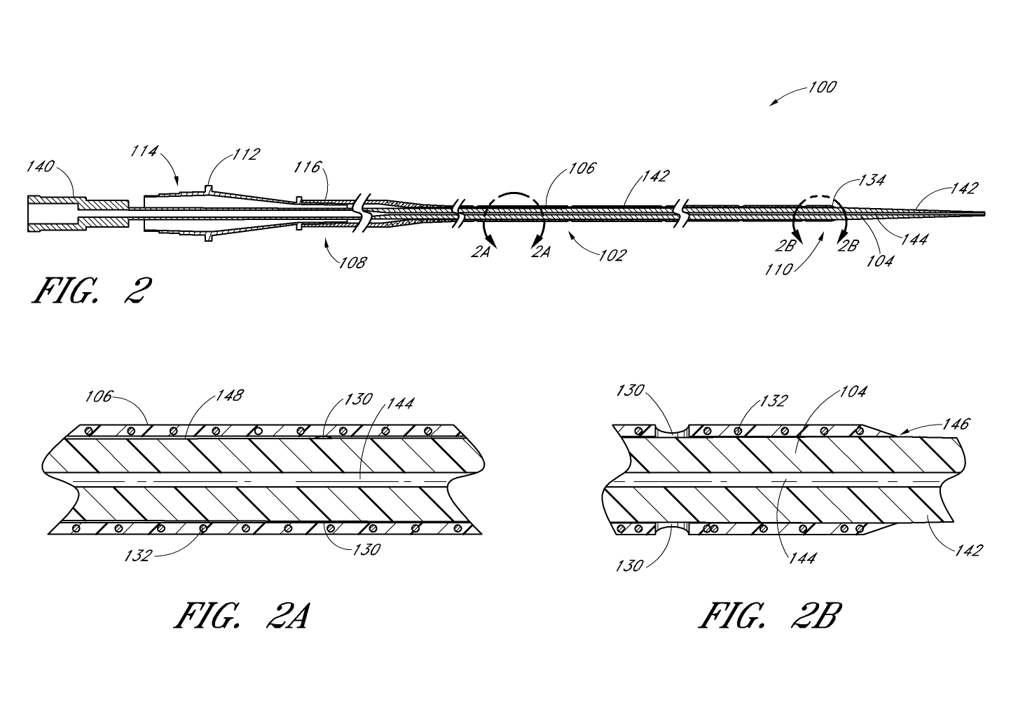

For example, the fourth segment 126 can be sized to provide an interference fit 146 (as shown in FIG. 2) between the cannula 102 and the obturator 104. The smaller diameters of the remaining segments 120, 122, 124, results in a small gap 148 between the cannula 102 and the obturator 104 (which is also illustrated in FIG. 2). In addition, the cannula 102 and obturator 104 are configured such that the cannula does not experience “fishmouthing” as the cannula system 100 is advanced through the tortuous vasculature of a medical patient. For example, the cannula 102 and obturator 104 are sized and configured such that they remain in contact with each other at the cannula 102 distal end as the cannula system 100 is advanced through tortuous vasculature. In addition the cannula 102 and obturator 104 can be sized and configured such that an opening, gap, or separation between the cannula 102 and obturator 104 does not occur at the cannula's distal end as the cannula system 100 is advanced through tortuous vasculature.

Certain segments can include reinforced regions 128 of greater stiffness than the flexible, elongate tube 106. The reinforced regions 128 also include one or more perfusion ports 130. The perfusion ports 130 may be used to remove or deliver fluids to the patient's vasculature. For example, in one embodiment, the cannula assembly 100 is used as a femoral venous cannula, and the distal end 110 of the cannula 102 is percutaneously delivered through a patient's femoral vein to the heart. The cannula 102 enters the right atrium of the heart via the inferior vena cava, and is advanced out of the heart via the superior vena cava. Deoxygenated blood returning to the heart via the inferior and superior vena cava is collected by the cannula's perfusion ports 130, and directed proximally through the cannula's elongate tube 106 to its proximal end. The deoxygenated blood is then delivered to a cardiopulmonary bypass device attached to the barbed end 114 of the cannula 102. Additional methods utilizing the features of the cannula assembly 100 are described in greater detail below.

In one embodiment, each reinforced region 128 includes four evenly-spaced perfusion ports 130 (e.g., each perfusion port 130 spaced from adjacent perfusion ports 130 by 90 degrees). In addition, all the perfusion ports 130 of a particular reinforced region 120 can be angularly offset by 45 degrees from all the perfusion ports 130 of an adjacent reinforced region 128. Such angular alignment advantageously provides improved fluid collection and disbursement.

A reinforcing coil 132 is provided along most of the length of the cannula's elongate tube 106. For example, in one embodiment, a reinforcing coil 132 extends from the cannula's tapered segment 118 almost to the end of its distal end 110. In another embodiment, the reinforcing coil 132 extends from the cannula's tapered segment 118 to the most proximal reinforced region 128, between reinforced regions 128, and from the most distal reinforced region 128 to or towards the cannula's distal end 110. The reinforced regions 128 are free of and do not include a reinforcing coil 132. The reinforcing coil 132 provides compression resistance, strength, and kink-resistance to the cannula's flexible, elongate body 106.

The distal end 110 of the cannula's elongate tube 106 terminates in a tapered portion 134. The tapered portion 134 provides a smooth transition from the outside surface of the cannula 102 to the outside surface of the obturator 104. The smooth transition allows the cannula assembly 100 to be inserted into and advanced through a patient's vasculature more easily and smoothly. A sharp or abrupt transition, such as from a step-down in diameter from the cannula to the obturator, could catch or snag on portions of the patient's vasculature, and could lead to tearing or other adverse clinical events during insertion and advancement therethrough.

The proximal end of the obturator 104 is formed as a handle 140. The handle 140 provides a convenient surface for holding and manipulating the obturator. The handle 140 has a larger diameter than the diameter of the cannula 102, and therefore functions as a stop to limit the relative movement of the obturator 104 with respect to the cannula 102.

The distal end 142 of the obturator 104 tapers to a reduced diameter. The distal end 142 taper allows the obturator 104 to function as a dilator, and facilitates the insertion and advancement of the cannula assembly 100 through the patient's vasculature. In addition, in one embodiment, the obturator 104 is made from a lubricious material, such as polytetrafluoroethylene (PTFE) or other plastic that further facilitates insertion and advancement of the cannula assembly 100 through the patient's vasculature.

An opening at the distal end of the obturator is sized to receive a guidewire. The cannula assembly 100 may therefore be advanced through a patient's vascular over a guidewire. The guidewire extends through an obturator lumen 144 (as can be seen in FIG. 2) from the obturator's handle 140 to its tapered distal end 142. The obturator 104 extends through the cannula's cannula lumen 142, which extends from its connector's barbed end 114 to its elongate tube's distal end 110.

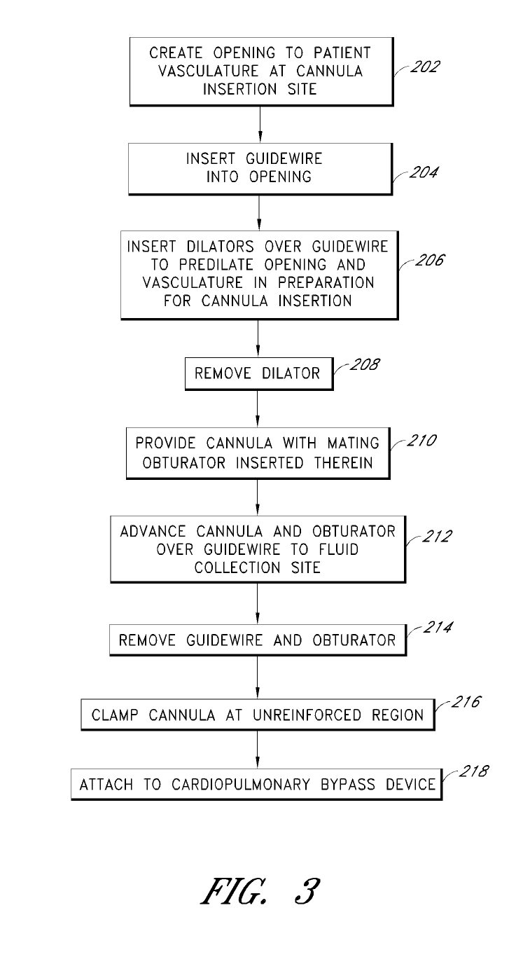

A method of deploying a cannula system in accordance with one embodiment of the present invention is illustrated in the flow chart of FIG. 3. The method 200 begins at block 202. At block 202, an opening to the patient's vasculature at a desired cannula insertion site is created. For example, a medical practitioner may create an incision in a patient's groin region to access the femoral vein. At block 204 a guidewire is inserted into the opening and advanced through the patient's vasculature to the desired fluid collection site. For example, in one embodiment, the guidewire is advanced through the patient's inferior vena cava and right atrium and into the superior vena cava.

At block 206, a series of dilators are inserted into the patient's vasculature over the guidewire to predilate the opening and vasculature and to prepare for cannula insertion. Multiple dilators of varying diameters may be utilized to dilate the opening into the patient's vasculature, as well as the vasculature itself. Because of the vasculature's elasticity, the final dilator is selected to have a diameter slightly larger than the diameter of the cannula.

At block 208, the dilator is removed. At block 210, a cannula with mating obturator is provided and inserted into the dilated opening. For example, in one embodiment, the cannula system 100 described above, is provided an inserted into the dilated vascular opening over the guidewire.

At block 212, the cannula system is advanced over the guidewire to a fluid collection site. For example, in one embodiment, the distal end of the cannula system is advanced over the guidewire until it reaches the superior vena cava of the patient's heart. When properly positioned, perfusion ports within the cannula wall are located in the patient's superior and inferior vena cava.

At block 214, the guidewire and obturator are removed from the cannula. As the guidewire and obturator are removed, the cannula may be clamped at an unreinforced region, as described in block 216. Such clamping minimizes blood leakage through the cannula as the obturator and guidewire are removed. At block 218, the proximal end of the cannula is attached to a device for fluid collection. For example, in one embodiment, the proximal end of the cannula is attached to the tubing of a cardiopulmonary bypass device.

In certain embodiments, features of the cannulae and related methods described above are applied to, or use in accordance with any one or more of the devices and methods described in U.S. Pat. Nos. 6,837,864 and 6,902,545, which are incorporated by reference in their entireties herein.

What is claimed is:

1. A cannula system for removing fluid from a patient's vasculature, comprising:
a cannula, the cannula comprising:
a proximal end;
a distal end; and
an elongate flexible tube extending therebetween and having a central lumen; and
an obturator, the obturator sized for insertion into the central lumen and configured to extend beyond the cannula distal end,
wherein a tapered portion at the cannula distal end provides a smooth transition and an interference fit between the cannula and the obturator, and
wherein the obturator includes an obturator central lumen and an opening at the obturator's distal end sized to receive a guidewire.

2. The cannula system of claim 1, wherein the elongate flexible tube further comprises:
a plurality of longitudinally spaced reinforced regions, each reinforced region comprising a plurality of perfusion ports; and
a plurality of reinforcing coils extending from a proximal portion of the elongate flexible tube to a first one of said reinforced regions, between reinforced regions, and from a last of said reinforced regions to a distal portion of the elongate flexible tube.

3. The cannula system of claim 2, wherein the reinforced regions include four perfusion ports.

4. The cannula system of claim 2, wherein the perfusion ports of adjacent reinforced regions are rotated forty-five degree about a central axis of the cannula with respect to each other.

5. The cannula system of claim 1, wherein the elongate, flexible tube and obturator define a gap therebetween at a proximal region of the cannula assembly.

6. The cannula system of claim 1, wherein the cannula mates with the obturator such that cannula and obturator are configured to remain in contact with each other at the tapered portion as the cannula system is advanced through a medical patient's vasculature.

7. The cannula system of claim 1, wherein the cannula does not experience fishmouthing as the cannula system is advanced through a medical patient's vasculature.

8. The cannula system of claim 1, further comprising a connector located at the elongate tube's proximal end.

9. The cannula system of claim 8, wherein the connector is sized and configured to be removably attached to a cardiopulmonary bypass device.

10. A method of deploying a cannula system within a medical patient's vasculature, comprising:
creating an opening to the medical patient's vasculature;
inserting a guidewire into the opening and through the vasculature to a deployment site;
dilating the opening and at least a portion of the vasculature by inserting a dilator over the guidewire;
removing the dilator from the vasculature and guidewire;
inserting a cannula system over the guidewire, through the opening and through the vasculature; and
advancing the cannula system through the vasculature over the guidewire and to the deployment site, the cannula system comprising a cannula and an obturator positioned within a central lumen of the cannula.

11. The method of claim 10, further comprising attaching a proximal end of the cannula system to a cardiopulmonary bypass device.

12. The method of claim 10, further comprising directing blood from the patient's vasculature to a cardiopulmonary bypass device with the cannula system.
